(19) United States
(12) Patent Application Publication
(10) Pub. No.: US 2008/0208205 A1
(43) Pub. Date: Aug. 28, 2008

(54) CABLE SYSTEM AND METHODS

(76) Inventor: Paul Edward Kraemer, Seattle, WA (US)

Correspondence Address: WILEY REIN LLP, 1776 K. STREET N.W., WASHINGTON, DC, 20006, US

(21) Appl. No.: 12/034,446

(22) Filed: Feb. 20, 2008

Related U.S. Application Data
(63) Continuation of application No. 12/033,350, filed on Feb. 19, 2008.
(60) Provisional application No. 60/903,347, filed on Feb. 26, 2007.
(60) Provisional application No. 60/903,411, filed on Feb. 26, 2007.

(51) Int. Cl. A61B 17/56

(52) U.S. Cl. 606/103; 606/60; 606/79

(57) ABSTRACT

A system for the percutaneous application of surgical wires or cables around bone includes a first flexible member and a second flexible member, each having an outer wall defining a passageway, an opening extending longitudinally along the outer wall and a curved distal portion terminating in a distal tip. The flexible members may also include a groove distal tips are shaped complementarily. The system also includes a stylet having a tension cable interconnecting a handle segment, a tip segment and one or more intermediate segments. The stylet is insertable through the passageways of the first and second flexible members to facilitate placement of the flexible members around bone. When joined at their distal tips, the first and second flexible members create a continuous path from the point of incision around bone. The first and second flexible members may be easily removed by pulling them apart and over the surgical wires or cables through the opening in the outer wall.

### CROSS-REFERENCE TO RELATED APPLICATIONS

This application is a continuation of U.S. application Ser. No. 12/033,350, filed Feb. 19, 2008, which claims the benefit of U.S. provisional application Nos. 60/903,347 and 60/903,411, both filed 26 Feb. 2007. The foregoing are hereby incorporated by reference as though fully set forth herein.

### BACKGROUND OF THE INVENTION

a. Field of the Invention

The present invention relates to a system for wrapping a wire or cable around an obstructed structure by accessing the structure through a small opening. In particular, the invention relates to a system for the percutaneous or near percutaneous application of surgical wires or cables in orthopedic fracture surgery and related methods. Even more particularly, the present invention relates to devices and methods for securing surgical wires or cables in such procedures.

b. Background Art

Cerclage cables or wires are used in bone fixation procedures, most commonly in periprosthetic fracture applications where there is an intramedullary stem or implant. Cerclage cables encircle a bone or structure and are cinched and clamped to hold portions of the bone together or retain an intramedullary stem or implant in place. The application of these cables typically requires a large incision to permit access to the fracture site and facilitate passing of the cable or wire around the intended structure. Current devices and methods for the application of cables or wires around bone often result in soft tissue damage, stripping, and devascularization at the site of the fracture. What is needed, therefore, is a system and methods for facilitating the passage of a cable or wire around bone through a small incision that minimizes or reduces these disadvantages.

### BRIEF SUMMARY OF THE INVENTION

The present invention meets these and other objectives by providing devices and methods for wrapping a wire or cable around a structure by accessing the structure through a small opening. In a first aspect, the present invention provides a system for passing wire or cable around bone that includes a first flexible member comprising an outer wall, the outer wall defining a first passageway extending through the first flexible member, an opening connected to the first passageway and extending longitudinally along the outer wall of the first member, a first curved distal portion terminating in a first distal tip, and a first groove extending longitudinally along at least a portion of the outer wall.

The system further includes a second flexible member comprising an outer wall, the outer wall defining a second passageway extending through the second flexible member, an opening connected to the second passageway and extending longitudinally along the outer wall of the second member, and a second curved distal portion terminating in a second distal tip. The second member is slidable along the first groove of the first member. In preferred embodiments, at least a portion of the second flexible member is shaped to fit within the first groove of the first flexible member. For example, the first groove may have a radius substantially equal to a radius of a circle defining an outer circumference of the second member. Alternatively, the outer wall of the second flexible member may be substantially complementarily shaped with the first groove.

In one aspect, a width of the opening of the first flexible member is approximately ½ to ¾ a thickness of the member. In another aspect, the opening is sufficiently wide to pass a surgical cable therethrough. The opening of the first and second flexible members extends longitudinally along the outer wall of each respective member from the proximal end up to and including the distal tip.

The first distal tip and the second distal tip of the first and second flexible members are preferably shaped complementarily. In one aspect, one of the first and second distal tips is concave and the other is convex. In another aspect, each of the first and second distal tips may include a magnet.

Preferably, the first curved distal portion and the second curved distal portion are each curved to wrap at least partially around a bone. In one embodiment, the first curved distal portion and the second curved distal portion may each be curved to wrap about 180 degrees around a bone.

In another embodiment, the second flexible member may include a second groove extending longitudinally along at least a portion of the outer wall of the second member, and at least a portion of the first flexible member may be shaped to fit within the second groove of the second flexible member.

In yet another embodiment, the system further includes a stylet insertable through the first and second passageways, the stylet including a plurality of segments and a tension cable interconnecting the plurality of segments, the plurality of segments being movable relative to one another when the tension cable is in a relaxed state and including at least a handle segment, a tip segment and one or more intermediate segments. The plurality of segments become substantially immovable relative to one another when the tension cable is tensioned. In one aspect, a distal end of the tip segment is beveled. In another aspect, a distal end of the tip segment includes a cutting surface.

In one embodiment, the stylet further comprises a lip between a most distal intermediate segment and the tip segment, and at least one of the first and second members further comprises a ridge near a distal end of the respective first or second passageway. The stylet is adapted to be advanced through the respective first or second passageway until the lip and the ridge abut one another. Preferably, no more than about 5-10 mm of the stylet protrudes out of the distal end of the respective first or second passageway when the lip and the ridge abut one another.

The present invention also provides an elongated member for passing wire or cable around bone, the member including a flexible body having a passageway therethrough, a curved distal portion terminating in a distal tip, an opening connected to the passageway extending longitudinally along an outer wall of the body, and a groove extending longitudinally along at least a portion of the outer wall of the body. The curved distal portion is curved to wrap at least partially around a bone, and a portion of the outer wall adjacent the groove is shaped complimentarily to the groove.

In one aspect, a width of the opening is approximately ½ to ¾ a thickness of the outer wall of the body. In another aspect, the groove has a radius substantially equal to a radius of a circle defining an outer circumference of the body.

The present invention further provides a method for passing a wire or cable around a structure, the method comprising: providing a first flexible member including a first passageway extending therethrough, a first curved distal portion terminating in a first distal tip, an opening extending longitudinally along an outer wall of the first member, and a groove extending longitudinally along at least a portion of the outer wall of the first member; wrapping the first curved distal portion of the first member at least partially around the structure; providing a second flexible member including a second passageway extending therethrough, a second curved distal portion terminating in a second distal tip, and an opening extending longitudinally along an outer wall of the second member, wherein at least a portion of the outer wall of the second member is shaped to fit within the groove of the first member; wrapping the second curved distal portion of the second member at least partially around the structure from the opposite direction as the first member; manipulating the first and second members to connect the first distal tip and the second distal tip; and, passing a wire or cable around the structure through the first and second passageways. The method further includes removing the first member by pulling the first member away from the wire or cable such that the opening extending along the outer wall of the first member passes over the wire or cable; and removing the second member by pulling the second member away from the wire or cable such that the opening extending longitudinally along the outer wall of the second member passes over the wire or cable.

In one aspect, the method further includes: providing a stylet having a handle at a proximal end thereof, a tip at a distal end thereof, one or more segments between the handle and the tip, and a tension cable interconnecting the handle, the tip and the one or more segments; prior to wrapping the first member at least partially around the structure, inserting the stylet through the first passageway and tensioning the tension cable until the stylet becomes rigid; and, after wrapping the first member at least partially around the structure, releasing tension of the stylet cable and removing the stylet from the first passageway.

In another aspect, the method further includes: prior to wrapping the second member at least partially around the structure, inserting the stylet through the second passageway and tensioning the tension cable until the stylet becomes rigid; and after wrapping the second member at least partially around the structure, releasing tension of the stylet cable and removing the stylet from the second passageway.

In preferred embodiments, the method includes placing the second member in the groove of the first member and sliding the second member along the groove to the near surface of the structure, such that the second member takes the same path as the first member to the near surface of the structure, prior to wrapping the second member at least partially around the structure.

In another preferred embodiment, the structure is a bone and the method includes: making an incision in a patient; inserting the first member through the incision prior to wrapping the first curved distal portion of the first member at least partially around the bone; and inserting the second member through the incision prior to wrapping the second curved distal portion of the second member at least partially around the bone. Typically, the method also includes locking the wire or cable after passing the wire or cable around the structure.

An advantage of the present invention is that it facilitates percutaneous or near percutaneous application of surgical wires or cables in orthopedic fracture surgery.

Another advantage of the present invention is that it minimizes the risk of damaging soft tissue structures, stripping, and devascularization at the site of the fracture.

A further advantage of the instant invention is that it facilitates easy removal of the flexible members over the wires or cables and crimping mechanism.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1A is a first flexible member according to one embodiment of the invention.

FIG. 1B is a second flexible member according to one embodiment of the invention.

FIG. 2 depicts first and second flexible members joined at their distal tips.

FIG. 3A illustrates a first flexible member having a concave distal tip and a second flexible member having a convex distal tip.

FIG. 3B depicts distal tips of first and second flexible members having complementary shapes according to one embodiment of the invention.

FIG. 4 illustrates a cross section of a flexible member having an opening in the outer wall.

FIG. 5A illustrates a cross section of a flexible member having an opening and a groove in the outer wall, the opening being located approximately 90 degrees from the groove.

FIG. 5B illustrates a cross section of a flexible member having an opening and a groove in the outer wall, the opening being located approximately 180 degrees from the groove.

FIG. 6 is a perspective view of a flexible member having an opening in the outer wall.

FIG. 7 is a perspective view of a flexible member having an opening and a groove in the outer wall.

FIG. 8 depicts a stylet according to one embodiment of the invention.

FIG. 9 illustrates the tip of a stylet protruding past the distal tip of a flexible member.

FIG. 10 is a schematic illustration of a cable clamp device according to an embodiment of the present invention.

FIG. 11 is a cross-section of the cable clamp device illustrated in FIG. 10 taken along line 11-11.

FIGS. 12-15 illustrate a sequence of steps that may be carried out in using a cable clamp device according to an embodiment of the present invention.

FIG. 16 illustrates a multi-use tool that may be used in accordance with an aspect of the present teachings.

### DETAILED DESCRIPTION OF THE INVENTION

The present invention provides devices and methods for passing wires or cables around a structure that is concealed or otherwise difficult to access. The present invention will be described in connection with the application of surgical wires or cables around bone in percutaneous or near percutaneous orthopedic fracture surgery. A person of skill in the art will appreciate, however, that the invention may be applied in other contexts where it is desirable to wrap a cable, wire or other elongated line, such as a rope or string, around a concealed or obstructed structure.

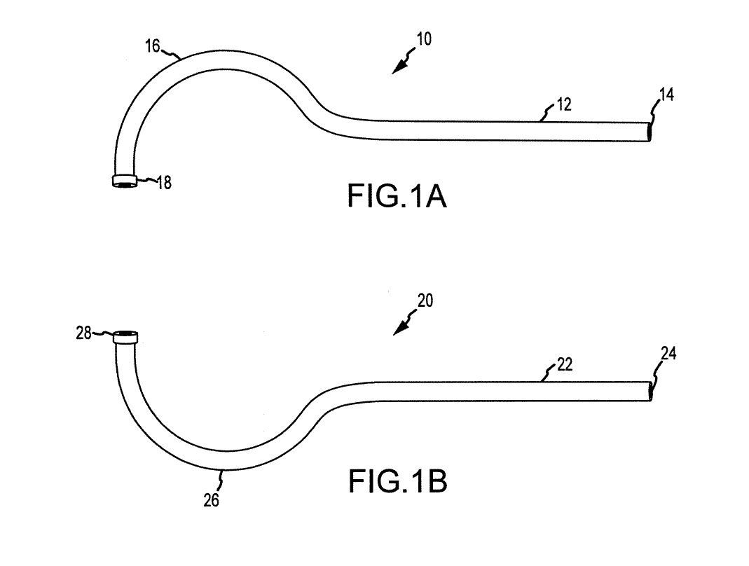

With reference to FIGS. 1A and 1B, a system according to one embodiment of the present invention is shown. FIG. 1A illustrates a first flexible member 10 and FIG. 1B depicts a second flexible member 20. Each of first and second flexible members 10, 20 includes an outer wall 12, 22 defining a passageway 14, 24 extending through the flexible member and a curved distal portion 16, 26 having a distal tip 18, 28. Flexible members 10, 20 have a generally tubular shape and are preferably made of a semi-rigid material, for example plastics. The diameter of passageways 14, 24 is sufficient for passing a surgical wire or cable therethrough.

Curved distal portions 16, 26 are shaped to curve at least partially around a bone. In preferred embodiments, curved distal portions 16, 26 of first and second flexible members 10, 20 are each shaped to curve approximately 180 degrees around a bone. In alternate embodiments, curved distal portion 16 of first flexible member 10 may be shaped to curve more than 180 degrees around a bone, and curved distal portion 26 of second flexible member 20 may be shaped to curve less than 180 degrees around the bone such that when joined at their distal tips first and second members 10, 20 substantially encircle the bone. For example, curved distal portion 16 of first flexible member 10 may be shaped to curve approximately 230 degrees around a bone and curved distal portion 26 of second flexible member 20 may be shaped to curve approximately 130 degrees around the bone. A person of skill in the art will appreciate that the shape of curved distal portions 16, 26 can be varied in complementary fashion so that the members substantially encircle the bone when joined at their distal tips. The radius of curvature of flexible members 10, 20 may also be varied to accommodate bones of different sizes.

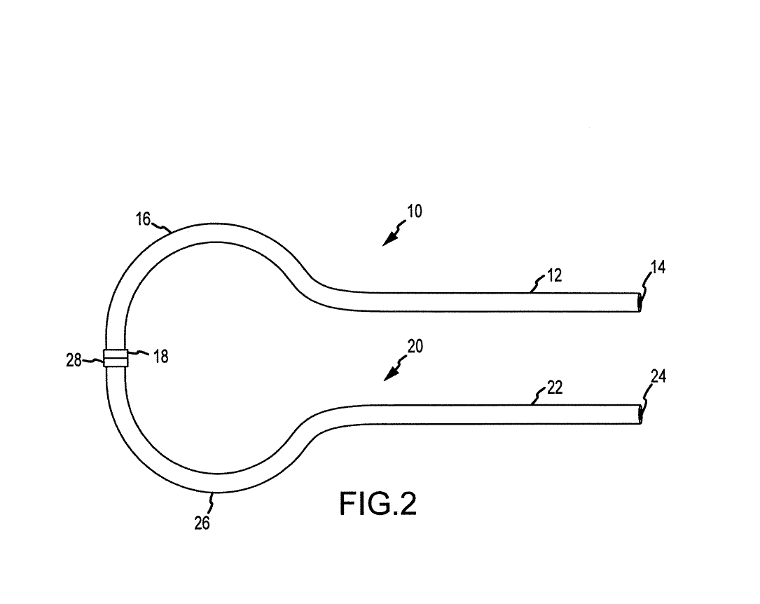

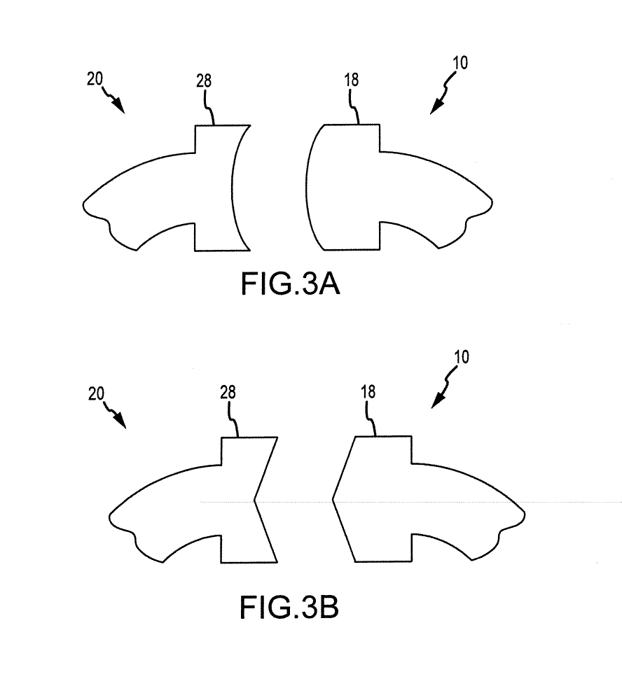

First and second flexible members 10, 20 are preferably used in a complementary fashion whereby each extends at least partially around a bone, as discussed above, and distal tips 18, 28 are joined or connected, as shown in FIG. 2. Preferably, distal tips 18, 28 are shaped complementarily. For example, distal tip 18 may be concave and distal tip 28 may be convex, as shown in FIG. 3A. Alternatively, distal tip 18 may be pointed and distal tip 28 may be indented, as shown in FIG. 3B. A person of skill in the art will appreciate that distal tips 18, 28 can be shaped in any complementary fashion such that distal tips 18, 28 may be joined together on the far side of a bone with minimal manipulation. Distal tips 18, 28 may alternatively include a magnet for connecting distal tips 18, 28 together. In embodiments in which distal tips 18, 28 include magnets, distal tips 18, 28 may be identically shaped, for example, distal tips 18, 28 may be flat. Distal tips 18, 28 are hollow or cannulated to facilitate the passing of a surgical wire or cable from one flexible member to the other. When distal tips 18, 28 are joined, a generally continuous passageway is formed from first flexible member 10 to second flexible member 20.

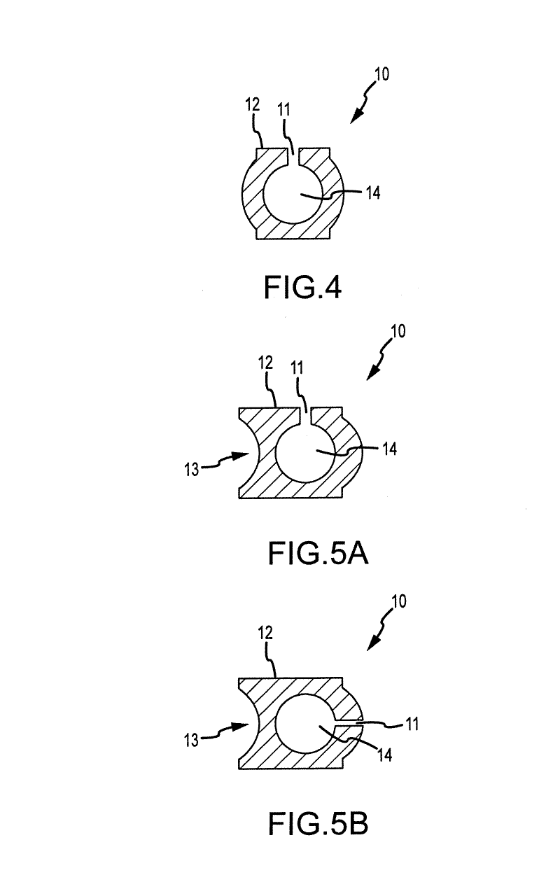

The following features will be discussed with reference to first flexible member 10, but it should be understood that these features may be present in second flexible member 20 without departing from the spirit and scope of the present invention. With reference to FIGS. 4-7, in preferred embodiments, first flexible member 10 includes an opening 11 connected to passageway 14 that extends longitudinally along outer wall 12. Opening 11 preferably extends longitudinally along outer wall 12 of flexible member 10 from a proximal end of the member through the distal tip. Once a surgical wire or cable is in place, flexible member 10 can be removed with minimal force by sliding or pulling it over the wire or cable. Because flexible member 10 is made of a semi-rigid material, opening 11 may stretch or flex to permit flexible member 10 to pass over large articles such as a surgical cable having a diameter larger than the width of opening 11 or a locking or cable crimp mechanism. The width of opening 11 is preferably approximately ½ to ¾ of the thickness of the outer wall at its most narrow surface. The opening 11 should be sufficiently wide to permit a surgical cable or wire to pass through opening 11 when removing flexible member 10, but sufficiently narrow to maintain the cable or wire within passageway 14 of flexible member 10 prior to removing the member.

With reference to FIGS. 5A-B and 7, first flexible member 10 preferably includes a groove 13 extending longitudinally along a portion of outer wall 12. Groove 13 serves as a track for the introduction of second flexible member 20 into the patient along the same soft tissue plane created with first flexible member 10. Groove 13 preferably extends along outer wall 12 to at least the proximal end of curved distal portion 16. Alternatively, groove 13 may extend along the full length of outer wall 12 to distal tip 18. Groove 13 is preferably shaped complementarily to at least a portion of outer wall 22 of second flexible member 20. In preferred embodiments, groove 13 is concave and at least a portion of outer wall 22 of second flexible member 20 is substantially congruent with the concavity of groove 13 such that outer wall 22 of second flexible member 20 fits within groove 13 of first flexible member 10 and is slidable along groove 13. Second flexible member 20 may also include a groove (not shown) in outer wall 22. The groove in outer wall 22 is preferably concave, and at least a portion of outer wall 12 of first flexible member 10 is substantially congruent with the concavity of the groove in second flexible member 20.

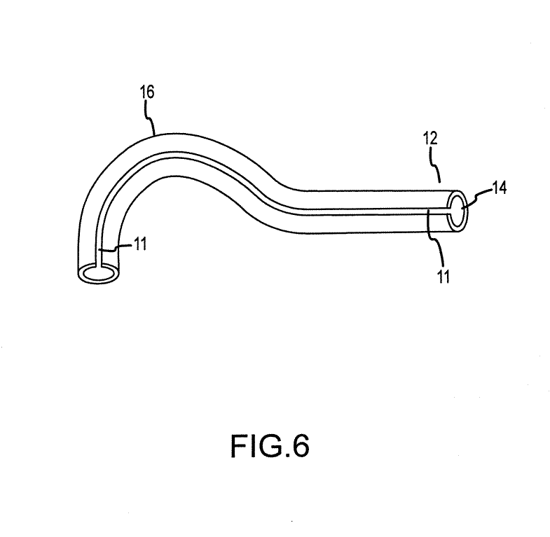

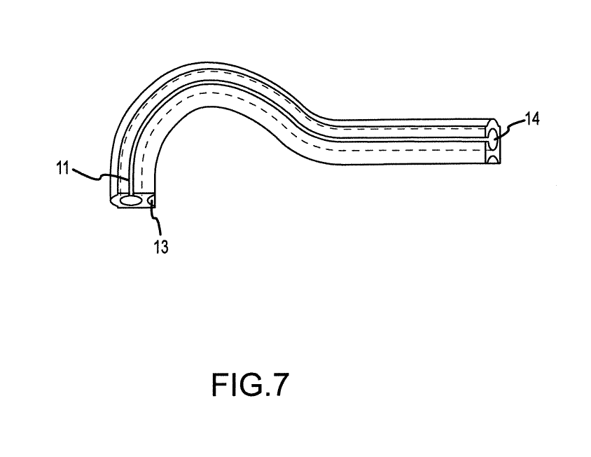

Opening 11 is preferably located on a portion of outer wall 12 of flexible member 10 that does not include groove 13. For example, in FIG. 5A, opening 11 is located approximately 90 degrees from groove 13. In one preferred embodiment, opening 11 is located approximately 90 degrees from groove 13 along the entire length of outer wall 12 and through distal tip 18. Alternatively, as shown in FIG. 5B, opening 11 is located approximately 180 degrees from groove 13 on a convex surface of outer wall 12. In another preferred embodiment, opening 11 begins on the convex surface of outer wall 12, as shown in FIG. 5B, and extends longitudinally along outer wall 12 of flexible member 10 until it reaches the proximal end of curved distal portion 16 (see FIGS. 6 and 7), at which point opening 11 is shifted approximately 90 degrees in either direction. This ensures that a cable or wire inserted through passageway 14 will have a solid surface to track against when passing through curved distal portion 16.

In preferred embodiments, first and second flexible members 10, 20 are identical. In other preferred embodiments, first and second flexible members 10, 20 may be identical except for the shape of distal tips 18, 28, which may be complementary. Preferably, at least one of flexible members 10, 20 includes a groove 13, but it should be understood that both flexible members may include a groove 13.

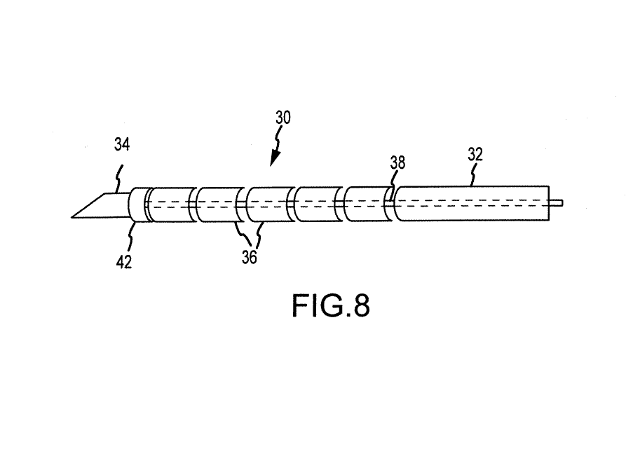

With reference to FIG. 8, a system according to one embodiment of the present invention further includes a stylet 30. Stylet 30 includes a handle 32 at the proximal end, a tip 34 at the distal end and one or more intermediate segments 36 located between handle 32 and tip 34. Stylus 30 is preferably made of a biocompatible metal such as, for example, stainless steel or titanium. In preferred embodiments tip 34 is beveled. More preferably, tip 34 includes a cutting surface, such as a blade or sharp edge. The cutting surface of tip 34 is preferably convex and congruent with the concavity of groove 13 (see FIG. 5). Accordingly, groove 13 may be used as a track to guide tip 34, as will be discussed in more detail herein. Cutting surface of tip 34 allows the stylus to cut a path very near the bone and avoid entrapment of adjacent structures or soft tissue. In addition, tip 34 may be used to facilitate joining or connecting first and second flexible members 10, 20, as described in more detail below.

Stylet 30 further includes a tension cable 38 that interconnects handle 32, intermediate segments 36 and tip 34. Handle 32, intermediate segments 36 and tip 34 are movable relative to one another when tension cable 38 is in a relaxed state, and become substantially rigid or immovable relative to one another when tension cable 38 is tensioned. Stylet 30 is insertable into passageways 14, 24 of flexible members 10, 20. After insertion, cable 38 is tensioned so that stylet 30 may conform to the shape of flexible members 10, 20.

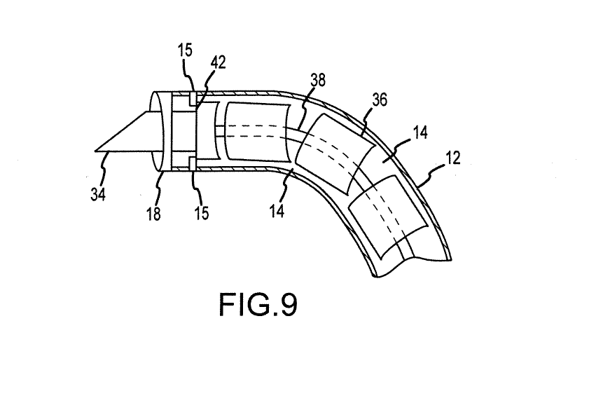

Referring now to FIGS. 8 and 9, in preferred embodiments stylet 30 also includes a lip 42 located between the distal-most intermediate segment 36 and tip 34. Lip 42 may be molded as a single piece with tip 34 or may be a separate piece that is coupled or otherwise attached to tip 34. A person of skill in the art will understand that there are multiple ways to form lip 42. Flexible member 10 may also comprise a ridge 15 near the distal end of passageway 14 (FIG. 9). When inserted into flexible member 10, for example, stylet 30 may be advanced until lip 42 abuts ridge 15. Preferably, ridge 15 is positioned such that tip 34 projects approximately 5-10 mm past distal tip 18 of flexible member 10 when lip 42 and ridge 15 abut. A person of skill in the art will appreciate, however, that the location of ridge 15 may be varied within passageway 14 to permit tip 34 to project less than 5 mm or more than 10 mm past distal tip 18 of flexible member 10. Similarly, the location of lip 42 on tip 34 may be varied to adjust the amount of protrusion of tip 34 from the distal end of flexible member 10. Although ridge 15 is described with reference to flexible member 10, it should be understood that a ridge may also be included in flexible member 20 as described herein without departing from the spirit and scope of the invention.

A method for passing a wire or cable around a structure, such as a bone, is now described. While the method is described in the context of orthopedic fracture surgery, a person of skill in the art will appreciate that the method may be useful in other contexts where it is desirable to wrap a line, such as a wire, rope or string, around a concealed or obstructed structure.

Stylet 30 is inserted into passageway 14 of first flexible member 10 until lip 42 abuts ridge 15. Tension cable 38 is tensioned so that handle 32, tip 34 and intermediate segments 36 become substantially rigid with respect to one another and stylet 30 conforms to the shape of first flexible member 10. First flexible member 10 and stylet 30 are inserted through a single incision to the near surface of a bone. When introducing first flexible member 10 and stylet 30, cutting surface of tip 34 may be used to cut a path very near the bone as described above to avoid entrapment of adjacent structures or soft tissue. First flexible member 10 is manipulated to wrap curved distal portion 16 at least partially around the bone, for example by using the cutting surface of tip 34 to “scrape” around the surface of the bone. Tension cable 38 is then loosened and stylet 30 is removed from passageway 14 of first flexible member 10.

Stylet 30 is inserted into passageway 24 of second flexible member 20. Tension cable 38 is tensioned so that handle 32, tip 34 and intermediate segments 36 become substantially rigid with respect to one another and stylet 30 conforms to the shape of second flexible member 20. Second flexible member 20 is preferably inserted through the same incision as first flexible member 10. Outer wall 22 of second flexible member 20 may be placed in groove 13 of first flexible member 10 and slid along groove 13 to the near surface of the bone. In this manner, second flexible member 20 takes substantially the same path as first flexible member 10 thereby minimizing trauma to the patient. Alternatively, tip 34 of stylet 30 may be placed in groove 13 to guide flexible member 20 to the near surface of the bone along substantially the same path as first flexible member 10. Second flexible member 20 is manipulated to wrap curved distal portion 26 at least partially around the bone from the opposite direction as first flexible member 10. First flexible member 10 and second flexible member 20 are manipulated until distal tips 18, 28 are joined or interlocked on the far side of the bone. Tip 34 of stylet 30 may be advanced through distal tip 28 of second flexible member 20 and into distal tip 18 of first flexible member 10 in a retrograde fashion to facilitate joining distal tips 18, 28 of first and second flexible members together. Tension cable 38 is then loosened and stylet 30 is removed from passageway 24 of second flexible member 20.

A surgical wire or cable is introduced through the proximal end of first flexible member 10, pushed through passageway 14 and into passageway 24 of second flexible member 20, then out the proximal end of second flexible member 20. First and second flexible members 10, 20 are removed by gently pulling them apart. Openings 11 will permit first and second flexible members 10, 20 to slide over the surgical wire or cable and any other instruments that may be present, such as a cable crimp mechanism. The cable or wire may then be tightened in place with instruments designed for percutaneous tightening of wires.

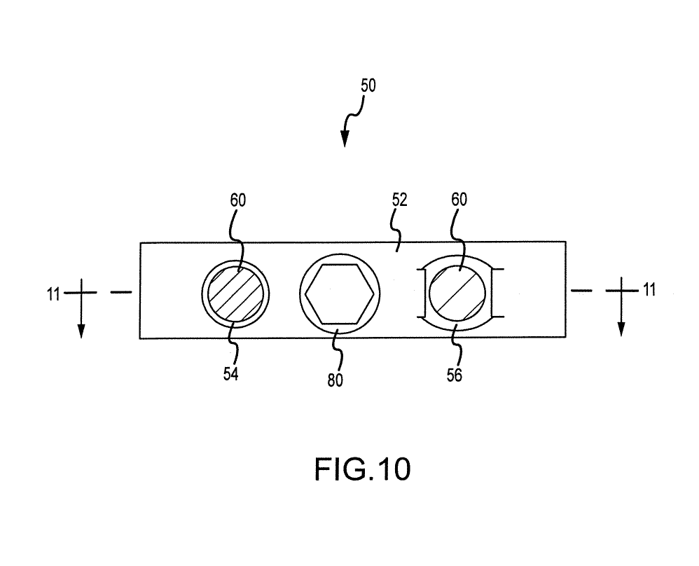

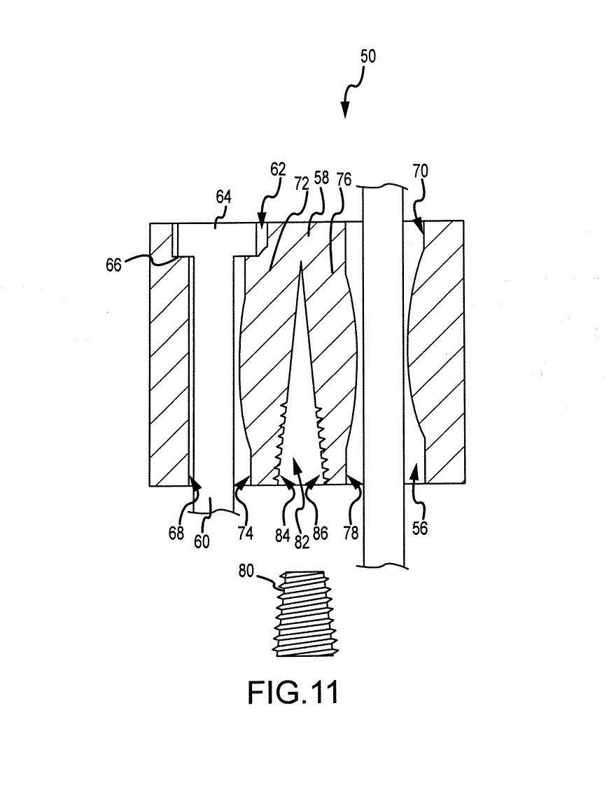

The present teachings also include a cable clamp device such as may be used to restrain the wire, cable, or other elongate member after it has been passed around a structure such as a bone. FIG. 10 schematically illustrates an embodiment of a cable clamp device 50 according to the present teachings. FIG. 11 is a cross-section of cable clamp device 50 taken along line 11-11 in FIG. 10 in order to illustrate various details of cable clamp device 50.

Cable clamp device 50 generally includes a housing 52 having substantially parallel first and second channels 54, 56, respectively, therethrough. At least one restraining member 58 is disposed within the housing between first channel 54 and second channel 56. Restraining member 58 is configured to restrain at least one elongate member, such as a surgical wire or cable 60 as illustrated in FIGS. 10 and 11, disposed within at least one of, and typically both of, first and second channels 54, 56. (For clarity, the intermediate portion of surgical wire or cable 60 is omitted in FIGS. 10 and 11.)

As illustrated in FIGS. 10 and 11, it is desirable for first channel 54, second channel 56, and restraining member 58 to be substantially coplanar and substantially parallel. This co-linear arrangement of channels 54, 56 and restraining member 58 is referred to herein as “side-loading” or “side-operated,” and is advantageous in percutaneous procedures generally, and particularly in percutaneous procedures through a single incision.

Each of first channel 54 and second channel 56 is shaped and dimensioned to pass surgical wire or cable 60 therethrough. It is contemplated that at least one channel (first channel 54 is illustrated) may also include an enlarged region 62 dimensioned to receive a cable head 64. Cable head 64 is larger than first channel 54 such that it cannot pass entirely therethrough. Instead, enlarged region 62 meets first channel 54 at a shoulder 66 upon which cable head 64 rides (that is, rests), thereby preventing surgical wire or cable 60 from pulling entirely through first channel 54 and releasing from cable clamp device 50. Of course, it is within the scope of the present invention for cable head 64 to ride on the outside of housing 52 instead of being recessed into housing 52 by virtue of enlarged region 62.

Preferably, restraining member 58 is a unitary expansion member disposed between first channel 54 and second channel 56. The term “expansion member” is used herein to refer to a member that can be increased in size (e.g., dilated), for example between a collapsed state and an expanded state. Thus, for example, when restraining member 58 is in its expanded state, a size of at least one of, and preferably both of, first and second channels 54, 56 is reduced as further described below.

In some embodiments of the invention, first channel 54 is defined in part by a first outer engagement surface 68, while second channel 56 is defined in part by a second outer engagement surface 70. Restraining member 58 similarly includes a first restraining arm 72 having a first inner engagement surface 74 and a second restraining arm 76 having a second inner engagement surface 78. First inner engagement surface 74 is opposite first outer engagement surface 68 and defines in part first channel 54. Similarly, second inner engagement surface 78 is opposite second outer engagement surface 70 and defines in part second channel 56.

The spacing between first restraining arm 72 and second restraining arm 76 is preferably adjustable via an actuator. In some embodiments of the invention, the actuator may be a tension screw 80 configured to be received within a tension screw receptacle 82 between first and second restraining arms 72, 76. Tension screw 80 may be wedge-shaped (e.g., conical or frusto-conical, referred to herein collectively as “conical”), cylindrical, or any other suitable shape. Preferably, first and second restraining arms 72, 76 are connected to each other opposite tension screw receptacle 82, though it is within the scope of the invention for first and second restraining arms 72, 76 to be disconnected from each other and “free-floating” between first and second channels 54, 56.

First restraining arm 72 includes a first locking surface 84 opposite first inner engagement surface 74 while second restraining arm 76 includes a second locking surface 86 opposite second inner engagement surface 78. Optionally, first and second locking surfaces 84, 86 may be inclined relative to each other such that as tension screw 80 is inserted or threaded into tension screw receptacle 82, first and second restraining arms 72, 76 are wedged apart, simultaneously reducing the size of first channel 54 and second channel 56. Of course, other configurations and arrangements of restraining member 58 and the associated actuator are contemplated.

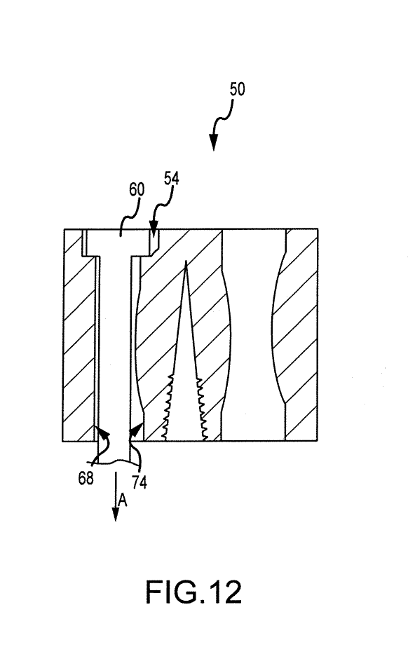

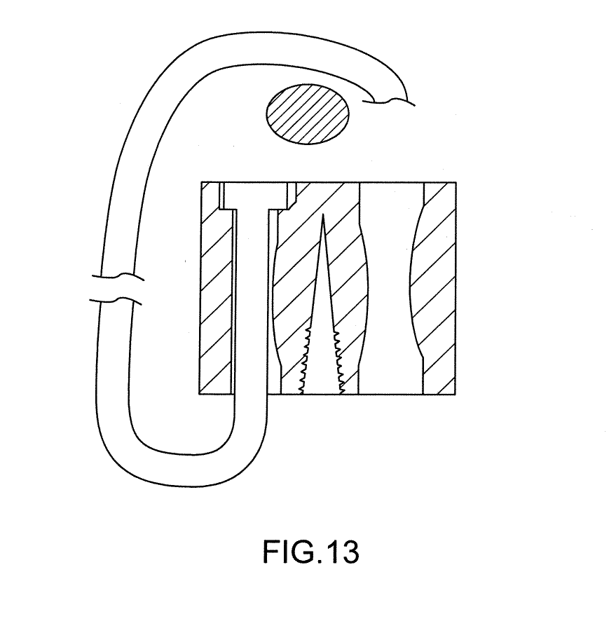

One exemplary use of cable clamp device 50 is illustrated in FIGS. 12-15. An elongate member, such as surgical wire or cable 60, is passed through first channel 54 until cable head 64 is riding on shoulder 66 such that surgical wire or cable 60 can be placed in tension. A first segment of surgical wire or cable 60 will be positioned within first channel 54 between first outer engagement surface 68 and first inner engagement surface 74. This is illustrated in FIG. 12.

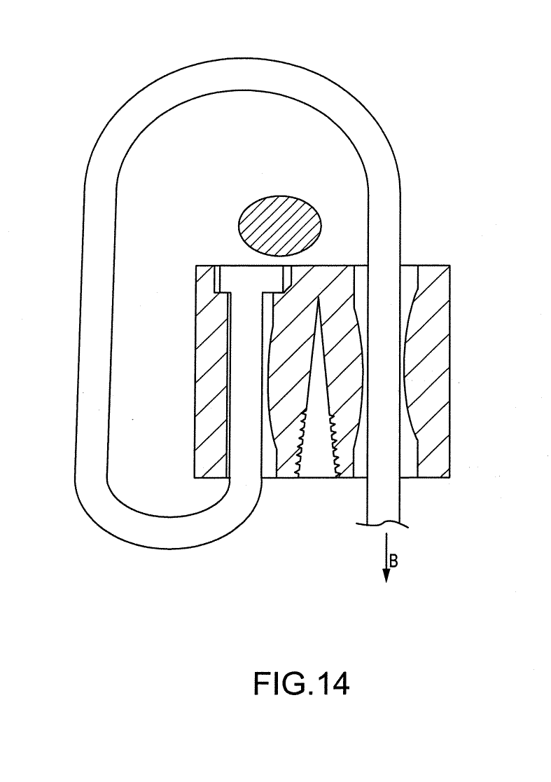

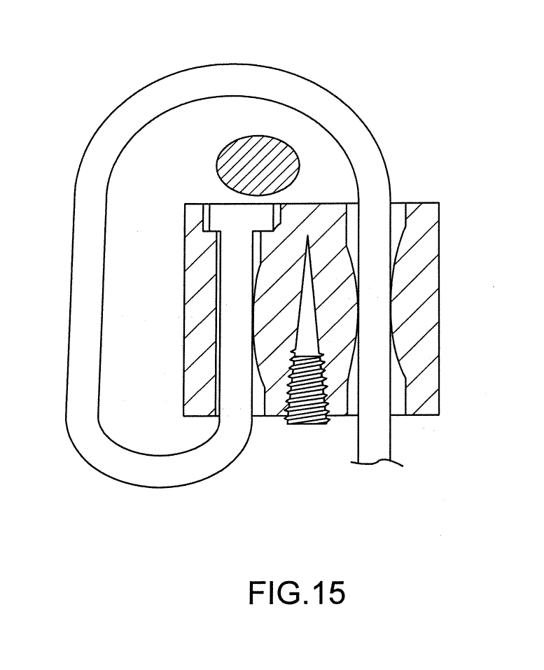

The elongate member is then passed around at least one structure to be bound (e.g., a bone or a bone and a plate), for example using the devices described above, as schematically illustrated in FIG. 13. (For clarity, the intermediate section of surgical wire or cable 60 is illustrated in FIGS. 13-15 as a thin line.) Once the elongate member has traveled the perimeter/circumference of the structure to be bound, it is passed through second channel 56 such that a second segment of the elongate member is positioned between second outer engagement surface 70 and second inner engagement surface 78. This is the configuration illustrated in FIG. 14. The elongate member may then be tensioned as desired by pulling in the direction of arrow “B” (e.g., by using the multi-use tool described below).

As illustrated in FIG. 15, restraining member 58 may then be actuated to impinge against the first segment of the elongate member within first channel 54 and the second segment of the elongate member within second channel 56. For example, a wedge, such as conical tension screw 80, may be driven into (e.g., threaded, as by use of the multi-use tool described herein) tension screw receptacle 82 between first and second restraining arms 72, 76. This restrains the elongate member from moving relative to cable clamp device 50, such as by simultaneously causing the first segment of the elongate member to be constrained between first restraining arm 72 and first outer engagement surface 68 and the second segment of the elongate member to be constrained between second restraining arm 76 and second outer engagement surface 70.

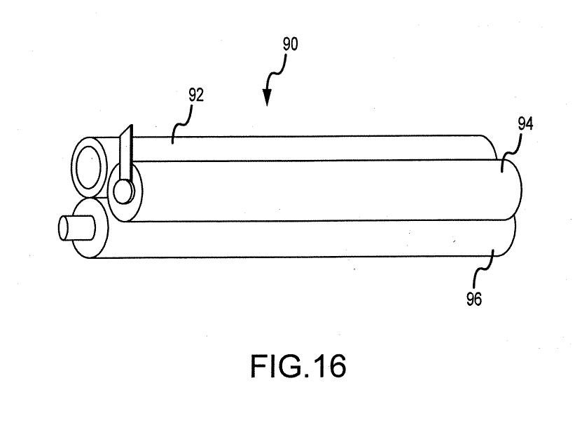

FIG. 16 illustrates a multi-use tool 90 that may be utilized to good advantage in conjunction with the cable passing and clamping devices described herein. Multi-use tool 90 generally includes a tensioning tool 92, a cutting tool 94, and an actuating tool 96. One of skill in the art will appreciate that multi-use tool 90 may include suitable handles (not shown) for utilizing tensioning tool 92, cutting tool 94, and actuating tool 96 as described below.

Tensioning tool 92 receives the free end of the elongate member after it has passed through second channel 56. Analogous to pushing a knot while suturing, tensioning tool 92 pushes cable clamp device 50 towards bone while pulling on the elongate member. In this fashion, tension may be applied to the elongate member until it is snugly around bone with cable clamp device 50 sitting on bone.

Actuating tool 96 is preferably positioned relative to tensioning tool 92 such that, when the elongate member is passed through tensioning tool 92, actuating tool 96 is aligned with tension screw 80. Actuating tool 96 is configured to mate with the actuator (e.g., it may include complementary devices, such as a hex-head screwdriver to mate with a hex-head tension screw 80 as illustrated in FIG. 10). Preferably, actuating tool 96 will be capable of a sufficient degree of translation within multi-use tool 90 without completely separating from multi-use tool 90. Of course, it is within the scope of the invention for actuating tool 96 to be separable from multi-use tool 90.

When the desired tension is achieved in the elongate member through the use of tensioning tool 92, actuating tool 96 may be used to actuate restraining member 58 to constrain the elongate member within first and second channels 54, 56. Cutting tool 94 may then be used to transect the elongate member at the level of cable clamp device 50.

Although several embodiments of this invention have been described above with a certain degree of particularity, those skilled in the art could make numerous alterations to the disclosed embodiments without departing from the spirit or scope of this invention. For example, it is contemplated that friction between the surgical cable or wire and the housing of the cable clamp device may be increased through the use of cleats, serrated nails, and the like.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A system for passing wire or cable around bone, the system comprising:
a first flexible member comprising
an outer wall, the outer wall defining a first passageway extending through the first flexible member,
an opening connected to the first passageway and extending longitudinally along the outer wall of the first member,
a first curved distal portion terminating in a first distal tip, and
a first groove extending longitudinally along at least a portion of the outer wall;
a second flexible member comprising
an outer wall, the outer wall defining a second passageway extending through the second flexible member,
an opening connected to the second passageway and extending longitudinally along the outer wall of the second member, and
a second curved distal portion terminating in a second distal tip,
wherein the second member is slidable along the first groove of the first member, and wherein the first distal tip and the second distal tip are shaped complementarily.

2. The system according to claim 1, wherein the first curved distal portion and the second curved distal portion are each curved to wrap at least partially around a bone.

3. The system according to claim 1, wherein the first curved distal portion and the second curved distal portion are each curved to wrap about 180 degrees around a bone.

4. The system according to claim 1, wherein one of the first and second distal tips is concave and the other is convex.

5. The system according to claim 1, wherein at least a portion of the second flexible member is shaped to fit within the first groove of the first flexible member.

6. The system according to claim 1, wherein the first groove is concave, and wherein the outer wall of the second flexible member is substantially congruent with the concavity of the first groove.

7. The system according to claim 1, wherein the second flexible member further comprises a second groove extending longitudinally along at least a portion of the outer wall of the second member, and wherein at least a portion of the first flexible member is shaped to fit within the second groove of the second flexible member.

8. The system according to claim 1, wherein a width of the opening of the first flexible member is approximately ½ to ¾ a thickness of the member.

9. The system according to claim 1, further comprising:
a stylet insertable through the first and second passageways, the stylet including a plurality of segments and a tension cable interconnecting the plurality of segments, the plurality of segments being movable relative to one another when the tension cable is in a relaxed state and including at least a handle segment, a tip segment and one or more intermediate segments,
wherein the plurality of segments become substantially immovable relative to one another when the tension cable is tensioned.

10. The system according to claim 9, wherein a distal end of the tip segment is beveled.

11. The system according to claim 9, wherein a distal end of the tip segment includes a cutting surface.

12. The system according to claim 9, wherein the stylet further comprises a lip between a most distal intermediate segment and the tip segment, wherein at least one of the first and second members further comprises a ridge near a distal end of the respective first or second passageway, and wherein the stylet is adapted to be advanced through the respective first or second passageway until the lip and the ridge abut one another.

13. The system according to claim 12, wherein no more than about 5-10 mm of the stylet protrudes out of the distal end of the respective first or second passageway when the lip and the ridge abut one another.

14. A system for passing wire or cable around bone, the system comprising:
a first flexible member comprising
a first passageway extending therethrough and including an opening through an outer wall of the first flexible member,
a first curved distal portion,
a first distal tip, and
a groove extending longitudinally along at least a portion of the outer wall of the first flexible member;
a second flexible member comprising
a second passageway extending therethrough and including an opening through an outer wall of the second flexible member,
a second curved distal portion, and
a second distal tip,
wherein at least a portion of the second flexible member is shaped to fit within the groove of the first flexible member; and
a stylet dimensioned to be insertable through the first and second passageways, the stylet including
a handle at a proximal end thereof,
a tip at a distal end thereof,
one or more segments between the handle and the tip, and
a tension cable interconnecting the handle, the tip, and the one or more segments,
wherein the stylet becomes substantially rigid when the tension cable is tensioned.

15. The system according to claim 14, wherein the first distal tip and the second distal tip are complementarily shaped.

16. The system according to claim 15, wherein one of the first and second distal tips is concave and the other is convex.

17. The system according to claim 14, wherein the first curved distal portion and the second curved distal portion are each curved to extend at least partially around a bone.

18. The system according to claim 17, wherein the first curved distal portion and the second curved distal portion are each curved to extend about 180 degrees around a bone.

19. The system according to claim 14, wherein the groove has a radius substantially equal to a radius of a circle defining an outer circumference of the second flexible member.

20. The system according to claim 14, wherein the opening through the outer wall of the first and second flexible members is sufficiently wide to pass a surgical cable therethrough.

21. The system according to claim 14, wherein the stylet tip is beveled.

22. The system according to claim 14, wherein the stylet tip includes a cutting surface.

23. An elongated member for passing wire or cable around bone, the member comprising:
a flexible body having a passageway therethrough,
a curved distal portion terminating in a distal tip,
an opening connected to the passageway extending longitudinally along an outer wall of the body, and
a groove extending longitudinally along at least a portion of the outer wall of the body,
wherein the curved distal portion is curved to wrap at least partially around a bone, and wherein a portion of the outer wall adjacent the groove is shaped complimentarily to the groove.

24. The member according to claim 23, wherein the distal tip comprises a magnet.

25. The member according to claim 23, wherein a width of the opening is approximately ½ to ¾ a thickness of the outer wall of the body.

26. The member according to claim 23, wherein the groove has a radius substantially equal to a radius of a circle defining an outer circumference of the body.

27. A method for passing a wire or cable around a structure, the method comprising:
providing a first flexible member including a first passageway extending therethrough, a first curved distal portion terminating in a first distal tip, an opening extending longitudinally along an outer wall of the first member, and a groove extending longitudinally along at least a portion of the outer wall of the first member;
wrapping the first curved distal portion of the first member at least partially around the structure;
providing a second flexible member including a second passageway extending therethrough, a second curved distal portion terminating in a second distal tip, and an opening extending longitudinally along an outer wall of the second member, wherein at least a portion of the outer wall of the second member is shaped to fit within the groove of the first member;
wrapping the second curved distal portion of the second member at least partially around the structure from the opposite direction as the first member;
manipulating the first and second members to connect the first distal tip and the second distal tip; and
passing a wire or cable around the structure through the first and second passageways.

28. The method according to claim 27, further comprising removing the first member by pulling the first member away from the wire or cable such that the opening extending along the outer wall of the first member passes over the wire or cable.

29. The method according to claim 27, further comprising removing the second member by pulling the second member away from the wire or cable such that the opening extending longitudinally along the outer wall of the second member passes over the wire or cable.

30. The method according to claim 27, further comprising:
providing a stylet having a handle at a proximal end thereof, a tip at a distal end thereof, one or more segments between the handle and the tip, and a tension cable interconnecting the handle, the tip and the one or more segments;
prior to wrapping the first member at least partially around the structure, inserting the stylet through the first passageway and tensioning the tension cable until the stylet becomes rigid; and,
after wrapping the first member at least partially around the structure, releasing tension of the stylet cable and removing the stylet from the first passageway.

31. The method according to claim 30, further comprising:
prior to wrapping the second member at least partially around the structure, inserting the stylet through the second passageway and tensioning the tension cable until the stylet becomes rigid; and
after wrapping the second member at least partially around the structure, releasing tension of the stylet cable and removing the stylet from the second passageway.

32. The method according to claim 27, further comprising:
placing the second member in the groove of the first member and sliding the second member along the groove to the near surface of the structure prior to wrapping the second member at least partially around the structure.

33. The method according to claim 27, wherein the structure is a bone.

34. The method according to claim 33, further comprising:
making an incision in a patient;
inserting the first member through the incision prior to wrapping the first curved distal portion of the first member at least partially around the bone; and
inserting the second member through the incision prior to wrapping the second curved distal portion of the second member at least partially around the bone.

35. The method according to claim 27, further comprising locking the wire or cable after passing the wire or cable around the structure.

36. A method of fixating a bone fracture, comprising:
making an incision in a patient;
providing a first flexible member including a first passageway extending therethrough, a first curved distal portion terminating in a first distal tip, an opening extending longitudinally along an outer wall of the first member, and a groove extending longitudinally along at least a portion of the outer wall of the first member;
inserting the first flexible member through the incision and wrapping the first curved distal portion of the first member at least partially around a bone in a first direction;
providing a second flexible member including a second passageway extending therethrough, a second curved distal portion terminating in a second distal tip, and an opening extending longitudinally along an outer wall of the second member, wherein at least a portion of the outer wall of the second member is shaped to fit within the groove of the first member;
inserting the second flexible member through the incision and wrapping the second curved distal portion of the second member at least partially around the bone in a second direction, the second direction being opposite of the first direction;
manipulating the first and second members to connect the first distal tip and the second distal tip;
passing a wire or cable around the bone through the first and second passageways;
removing the first member by pulling the first member away from the wire or cable such that the opening extending along the outer wall of the first member passes over the wire or cable; and
removing the second member by pulling the second member away from the wire or cable such that the opening extending longitudinally along the outer wall of the second member passes over the wire or cable.

37. The method according to claim 36, further comprising:
providing a stylet having a handle at a proximal end thereof, a tip at a distal end thereof, one or more segments between the handle and the tip, and a tension cable interconnecting the handle, the tip and the one or more segments;
prior to wrapping the first member at least partially around the bone, inserting the stylet through the first passageway and tensioning the tension cable until the stylet becomes rigid; and
after wrapping the first member at least partially around the bone, releasing tension of the stylet cable and removing the stylet from the first passageway.

38. The method according to claim 37, further comprising:
prior to wrapping the second member at least partially around the bone, inserting the stylet through the second passageway and tensioning the tension cable until the stylet becomes rigid; and
after wrapping the second member at least partially around the bone, releasing tension of the stylet cable and removing the stylet from the second passageway.

39. The method according to claim 38, wherein the stylet tip protrudes no more than about 5 mm out of the second distal tip of the second flexible member when inserted through the second passageway, the stylet tip being adapted to engage the first distal tip of the first flexible member to connect the first and second flexible members.

40. The method according to claim 39, wherein the stylet tip passes through the first distal tip in a retrograde fashion to engage the first distal tip.

41. The method according to claim 36, further comprising:
placing the second member in the groove of the first member and sliding the second member along the groove to the near surface of the bone prior to wrapping the second member at least partially around the bone.

42. The method according to claim 36, further comprising locking the wire or cable after passing the wire or cable around the bone.
